(19) United States
(12) Patent Application Publication
(10) Pub. No.: US 2009/0082689 A1
(43) Pub. Date: Mar. 26, 2009

(54) METHOD AND APPARATUS FOR REDUCING THE NUMBER OF CHANNELS IN AN EEG-BASED EPILEPTIC SEIZURE DETECTOR

(76) Inventors: John V. Guttag, Lexington, MA (US); Ali Shoeb, Winchester, MA (US); Elena L. Glassman, Pipersville, PA (US); Eugene I. Shih, Cambridge, MA (US)

Correspondence Address: K&L Gates LLP, STATE STREET FINANCIAL CENTER, One Lincoln Street, BOSTON, MA, 02111-2950, US

(21) Appl. No.: 12/196,690

(22) Filed: Aug. 22, 2008

Related U.S. Application Data
(60) Provisional application No. 60/965,890, filed on Aug. 23, 2007.

(51) Int. Cl. A61B 5/0478

(52) U.S. Cl. 600/544

(57) ABSTRACT

An ambulatory patient-specific epileptic seizure detector based on scalp EEG signals is presented. A method for selecting a patient-specific subset of electrodes from a plurality of m EEG channels needed to detect an epileptic seizure in the patient is also presented. Seizure EEG data is collected from the plurality of m EEG channels. An effective subset n of the channels of the plurality of m EEG channels is selected using recursive feature processing and a detector is constructed in response to the subset n of channels. The performance of the detector in detecting seizures is then estimated.

### CROSS-REFERENCE TO RELATED APPLICATIONS

This application claims the benefit of U.S. Provisional patent application No. 60/965,890 filed Aug. 23, 2007, the entire disclosure of which is incorporated herein by reference.

### STATEMENT OF GOVERNMENT INTEREST

This invention was made with Government support under U.S. Army Grant No. DAMD-17-02-2-0006.

### BACKGROUND OF THE INVENTION

Detecting the electrical onset of epileptic seizures using scalp electroencephalogram (“EEG”) can facilitate numerous diagnostic, therapeutic, and alerting applications. In some instances, seizure detection is used to initiate neuroimaging studies, such as Ictal SPECT, soon after the electrical onset of a seizure. The fidelity with which Ictal SPECT defines the cerebral origin of a seizure is enhanced by shortening the delay between seizure onset and the start of the study. Seizure onset detection is also used to trigger neurostimulators, such as the Vagus Nerve Stimulator, soon after the onset of a seizure. The likelihood of affecting the progression of a seizure using vagus nerve stimulation seems to decrease the longer the delay between the onset of a seizure and the start of stimulation. Additionally, seizure onset detection can prompt an individual to seek safety or self-administer a fast-acting anticonvulsant; this is possible in individuals for whom the electrical onset of a seizure and the start of physically debilitating symptoms are sufficiently separated in time. While the above-mentioned applications vary in utility and purpose, they all require detecting the electrical onset of seizures with minimum latency, high sensitivity, and high specificity; doing so, however, has proved to be a difficult task.

Robust detection of the onset of a seizure from scalp EEG is challenging for three primary reasons. First, variability exists in both the seizure (ictal) and non-seizure (interictal) EEG of different individuals. Second, for any given individual, some non-seizure activity (interictal epileptiform bursts) may closely resemble the seizure onset. Finally, scalp EEG is easily corrupted by both physiologic and non-physiologic artifacts.

Numerous algorithms for detecting seizure onset from scalp EEG have been proposed. These algorithms fall under the two broad categories of “patient-specific” and “patient non-specific” algorithms. Researchers developing patient non-specific algorithms sacrifice performance for the practicality of having an algorithm that is ready for use on any individual at any time. In contrast, investigators developing patient-specific methods incur the cost of collecting training data because they believe the consistency and relative separability of an individual's seizure and non-seizure EEG can be exploited for the purpose of enhancing performance. Quantifying the degree to which patient-specificity impacts the performance of a seizure onset detector will shed light on these trade-offs.

Further, ambulatory, patient-specific, epileptic seizure detectors require the use of cumbersome devices having up to twenty-one electrodes affixed to the patient at all times in order to detect seizure onset, and batteries sufficient to collect and process the signals from those electrodes. One such ambulatory system detects seizure onset using a detector that includes a cap with twenty-one EEG channels, the hardware needed to capture and process those channels, and the battery needed to power the hardware. Such devices utilize machine learning and support vector machines that produce patient-specific detectors with excellent sensitivity, specificity, and latency for most patients when used with full twenty-one-channel EEG montages. The cap which is to be worn at all times by the patient, however, is cumbersome and intrusive. If the number of channels were significantly reduced the system could be made considerably less burdensome on the patient and the requisite analysis in detecting seizure onset. Reducing the number of channels would also reduce the amount of energy needed to acquire and process the data, thus reducing the size or prolonging the life of batteries.

The present invention addressed these issues.

### SUMMARY

Embodiments of the present invention include methods and systems for reducing the number of channels in an EEG-based epileptic seizure detector. According to one embodiment, a method for selecting a patient-specific subset of electrodes from a plurality of m EEG channels needed to detect an epileptic seizure in the patient is presented. The method involves collecting seizure EEG data from the plurality of m EEG channels then selecting an effective subset n of the channels of the plurality of m EEG channels. A detector is constructed in response to the subset n of channels and the performance of the detector in detecting seizures is estimated.

A further aspect of the invention includes selecting the effective subset n of the channels by constructing a detector using the plurality of m channels using recursive feature elimination and estimating the performance of the detector. A least useful channel is removed from the plurality of m channels and the performance of the remaining plurality of channels is estimated. Removing a least useful channel and estimating the performance of the remaining channels is repeated until the performance of the remaining plurality of channels is worse than the performance of the previous plurality of channels. n is then set equal to the number of channels in the plurality of channels equal to one more than the number of channels that caused the performance of the remaining plurality of channels to be degraded.

A further aspect of the illustrative embodiment includes using recursive feature addition to construct a detector using the plurality of m channels and estimating the performance of the detector. To determine the best channel subset of size n, a set S is initialized to the best channel subset of size n−1. A most useful channel is added from the plurality of m channels. The most useful channel is determined by estimating the performance of the detector constructed using the best channel subset of size n−1 and this channel. The best channel subset of size 0 is the empty subset. This procedure is repeated until a stopping criterion has been met. One criterion may include when the performance of a particular subset of channels is no worse than the performance of the detector using the plurality of m channels. Alternatively, the procedure can be repeated until m channel subsets have been determined. A most useful channel subset is then selected from the m channel subsets based on maximizing a specific objective function.

Another embodiment includes a patient-specific epileptic seizure detector comprising a plurality of electrodes corresponding to a plurality of m EEG channels. A processor is configured to select a subset n of the channels of the plurality of m EEG channels using recursive feature elimination. The detector is constructed in response to the subset n of channels. An estimator is configured to estimate the performance of the detector in detecting seizures.

Features of the embodiment include a detector in which the subset n comprises the plurality of m channels minus a plurality of least useful channels. The least useful channels are determined by recursively removing the least useful channel from the plurality of m channels and estimating the performance of the remaining plurality of channels until the performance of the remaining plurality of channels is worse than the performance of the plurality of m channels. The subset n is equal to the number of channels in the plurality of channels equal to one more than the number of channels in the plurality of channels that caused the performance of the remaining plurality of channels to be worse than the performance of the plurality of m channels.

Yet another embodiment includes a patient-specific epileptic seizure detector comprising a plurality of electrodes corresponding to a plurality of m EEG channels. A processor is configured to select a subset n of the channels of the plurality of m EEG channels using recursive feature addition. The detector is constructed in response to the subset n of channels. An estimator is configured to estimate the performance of the detector in detecting seizures.

Features of the embodiment include a detector in which the subset n comprises the plurality of m channels minus a plurality of least useful channels. The subset n is determined by recursively adding a most useful channel incrementally to a previously determined best channel subset. This procedure is repeated until a stopping criterion has been met. One criterion may include when the performance of a particular subset of channels is no worse than the performance of the detector using the plurality of m channels. Alternatively, the procedure can be repeated until m channel subsets have been determined. A most useful channel subset is then selected from the m channel subsets based on maximizing a specific objective function.

### BRIEF DESCRIPTION OF THE DRAWINGS

These embodiments and other aspects of this invention will be readily apparent from the detailed description below and the appended drawings, which are meant to illustrate and not to limit the invention, and in which:

FIG. 1 is a diagram of the processing stages of a binary, patient-specific detector in accordance with an embodiment of the invention;

FIG. 2 is a graph of a feature extraction filterbank in accordance with an embodiment of the invention;

FIG. 3 is a diagram of the processing stages of a unary, patient-specific detector in accordance with an embodiment of the invention;

FIG. 4 is a table of EEG data set characteristics in accordance with an embodiment of the invention;

FIGS. 5-10 are graphs of performance comparisons of detector types;

FIGS. 11-12 depict the electrographic seizure states associated with the detection latency in accordance with embodiments of the invention;

FIG. 13 is a flow diagram depicting a method of choosing a subset of channels in accordance with an embodiment of the invention;

FIG. 14 is a table representing the performance results of a detector in accordance with an embodiment of the invention;

FIG. 15 is a series of histograms representing the channels chosen during a selection process in accordance with an embodiment of the invention;

FIG. 16 depicts a portion of a seizure detected from an EEG of a patient in accordance with an embodiment of the invention;

FIG. 17 depicts a portion of a seizure detected from an EEG of another patient in accordance with an embodiment of the invention; and

FIG. 18 represents an output of an EEG detector in accordance with an embodiment of the invention.

### DETAILED DESCRIPTION

Embodiments of the invention include methods and apparatus for detecting seizures. A first detector is trained on examples of both seizure and non-seizure EEGs from a test individual and is referred to herein as a binary patient-specific detector. A second detector is trained only on examples of non-seizure EEGs from the test individual and is referred to as the unary patient-specific detector. A third detector is not trained on any EEG from the test individual, and is referred to herein as the patient-specific detector.

### Detection Methods

EEG is an electrical record of brain activity that is collected using an array of electrodes distributed on a subject's scalp or inter-cranially. A channel is defined as the difference in potential recorded between a pair of (typically adjacent) electrodes or an electrode and a reference electrode.

### Binary

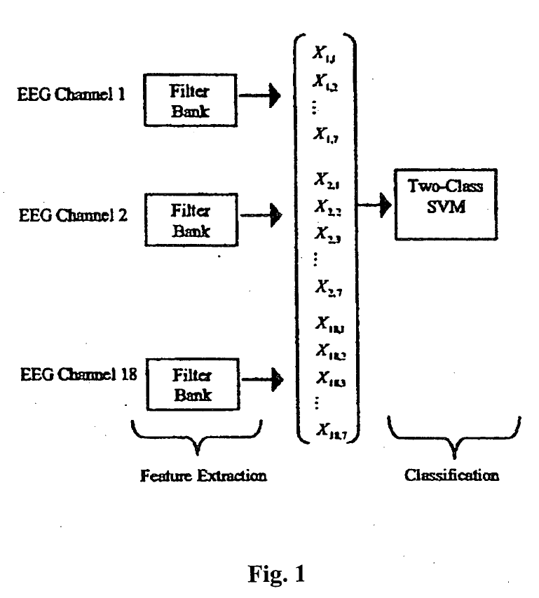

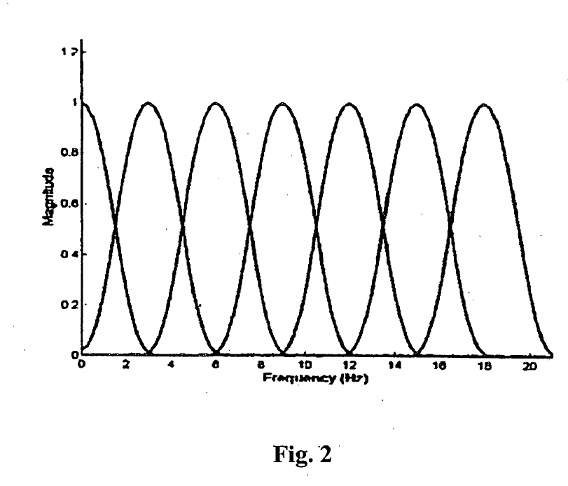

Turning now to FIG. 1, the processing stages of a binary patient-specific detector are illustrated. In this example the data is acquired through eighteen channels. The binary detector passes two-second epochs from each of eighteen EEG channels through a feature extractor. In turn, the feature extractor assembles, for each channel, a feature vector whose seven elements correspond to the energies in the seven frequency bands provided by the filter bank shown in FIG. 2. These frequency bands collectively cover the frequency range within which physiologic and pathophysiologic scalp EEG activity is observed.

The elements or features extracted from each of the eighteen channels are then concatenated to form a feature vector that captures spatial correlations between channels. The resulting feature vector is assigned to a seizure or a non-seizure class using a two-class support-vector machine (“SVM”) classifier trained on non-seizure EEG data (awake, sleep, interictal epileptiform bursts) and seizure onset EEG data from the same individual. According to one embodiment, the binary detector declares seizure onset when four seconds of EEG activity are classified as being consistent with the individual's seizure onset EEG data.

According to one embodiment, an SVM package such as the toolbox package by Anton Schwaighofer of Microsoft Research, Cambridge, UK, or the SVM^{light} software package by Thorsten Joachims, Department of Computer Science, Cornell University, Ithaca, N.Y., may be used to implement the two-class support-vector machine used in the binary patient-specific detector. According to one embodiment a radial-basis kernel with kernel parameter γ=1, and a trade-off between training error and margin C=10 for both the seizure and non-seizure classes was used.

### Unary

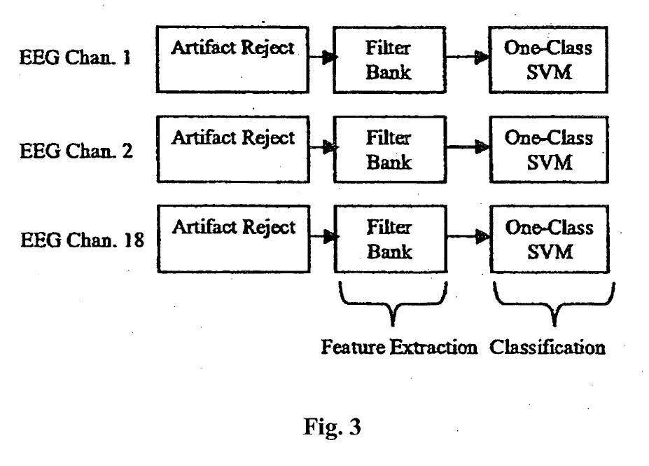

The block diagram in FIG. 3 illustrates the processing stages of a unary patient-specific detector. The unary detector uses standard techniques to reject any input channel whose two-second epoch is contaminated by an artifact. The unary detector assembles, for each artifact-free channel, a feature vector whose elements correspond to the energies in the frequency bands again using the filter bank shown in FIG. 2. The unary detector then uses a one-class SVM classified to determine whether the feature vector from each channel is consistent with the training non-seizure EEG data from the same channel. Seizure onset is declared if any channel exhibits activity inconsistent with the non-seizure training data for a duration of seven seconds. In a different embodiment a seizure is declared only if the selected seven second epoch conforms to non-patient specific criteria of eleptiform activity.

The LIBSVM software package, by Chih-Chung Chang and Chih-Jen Lin, Department of Computer Science and Information Engineering, National Taiwan University, Taipei, Taiwan, in one embodiment was used to implement the one-class SVM. In this embodiment, a radial-basis kernel has a kernel parameter γ=7 and a support-vector fraction v=0.0075.

In one embodiment the intracranial EEG features (e.g., mean Curve Length, mean Energy, mean Teager Energy) typically in a one-class SVM to detect seizure onset used for the purpose of detecting/predicting seizure onsets in intracranial EEGs (A. Gardner, A. M. Krieger, G. Vachtsevanos, B. Litt. “One-Class Novelty Detection for Seizure Analysis from Intracranial EEG.” Journal of Machine Learning Research 7 (200): 1025-1044) were replaced by the spectral energy features computed using the filter bank in FIG. 2. The spectral energy features yielded low detection latency and high specificity on the scalp EEG dataset used. In one embodiment automatic artifact rejection, processing all available EEG channels as opposed to only the channels on which a seizure is known to occur, and evaluating the modifications on continuous, scalp EEG recordings that include both awake and sleep periods were included in the processing.

### Patient Non-Specific

The patient non-specific detector used in one embodiment was a commercially available implementation known as the Reveal algorithm. The Reveal algorithm decomposes two-second EEG epochs from each input channel into time-frequency atoms using the Matching Pursuit algorithm, as detailed in “Seizure Detection: Evaluation of Reveal Algorithm” by Wilson, Scheuer, Emerson, Gabor in Clinical Neurophysiology 2004 October; 115(10):2280-91. Reveal then employs hand-coded and neural network rules to determine whether features derived from the time-frequency atoms of a channel are consistent with a seizure taking place on that channel. The thresholds for some of the neural network rules are determined using both archetypal seizures as well as non-seizure epochs from patients without epilepsy; no data from the test individual is used to tune the Reveal algorithm.

According to one embodiment, the Reveal algorithm was set to declare a seizure whenever a fifteen second segment was classified as being part of a seizure at a ninety-five percent (95%) confidence level. The typical default detector configuration with twenty second segments, and a fifty percent confidence level produces an unacceptable number of false detections.

### Evaluation

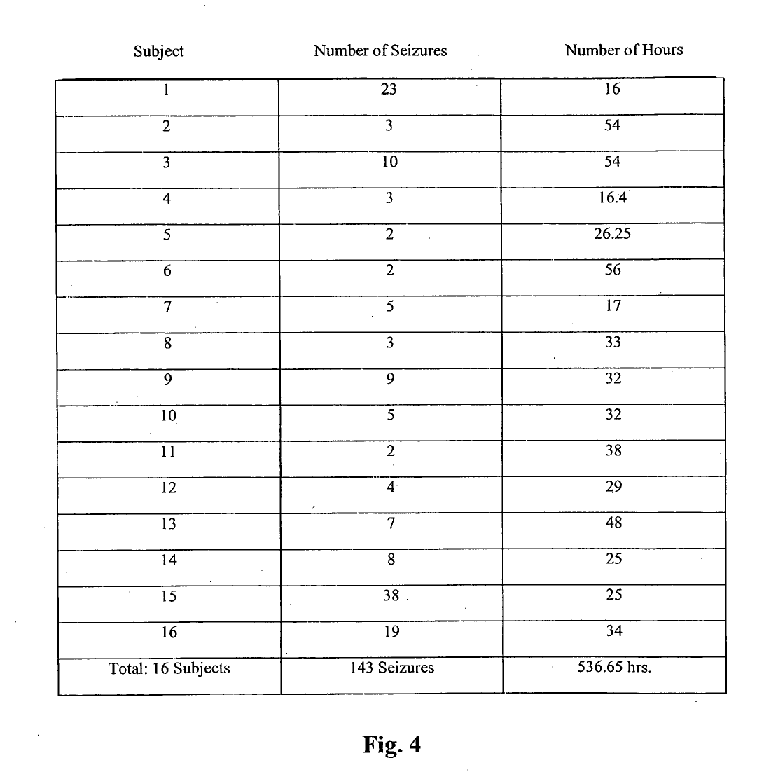

In testing embodiments of the invention, scalp EEG from pediatric inpatients at the epilepsy monitoring unit of Children's Hospital Boston was used to test the three seizure detection methods described above. The EEG was sampled at two-hundred fifty-six (256) Hz and recorded using an 18-channel bipolar montage. Overall, the test set (FIG. 4) contained 536 hours of continuously recorded EEG from sixteen subjects. For each subject, both awake and sleep EEG periods were recorded.

All SVM parameters in the detection methods were determined at the start of testing, and held constant over the course of all tests. The EEG data belonging to each patient was organized into consecutive, one-hour records. N denotes the number of seizure-free, one-hour records for a given patient and M denotes the number of one-hour records containing one or more seizures for a given patient. The performance of the patient non-specific detector on the N+M records of each patient was evaluated. The number of seizures missed; the average delay in declaring the electrical onset of detected seizures and the number of false detections were noted.

The performance of the binary patient-specific detector using two studies was then evaluated. In the first study the detector was trained on the N non-seizure records of the patient as well as M−1 records containing a seizure. The detector was then tasked with detecting seizures in the M^{th} seizure record; the record that was withheld from the training set. This process was repeated M times so that each of the M seizure records was tested once. A seizure record M was never simultaneously in the training and testing sets.

In the second study, the binary patient-specific detector was trained on the M seizure records of a patient as well as N−1 non-seizure records. The detector was then tasked with processing the N^{th} non-seizure record; the record that was withheld from the training set. This process was repeated N times so that each of the N non-seizure records is tested once; a non-seizure record N never simultaneously in the training and testing sets. Upon completion of these two tests, the binary patient-specific detector was tested on all the N+M records of the patient. The number of seizures missed, the average delay in declaring the electrical onset of detected seizures, and the number of false detections was noted.

For the unary patient-specific detector another pair of studies was conducted. In the first study the detector was trained on the N non-seizure records of a patient. The detector is then tasked with detecting seizures in the M seizure records withheld from the training set.

In the second study the unary patient-specific detector was trained on N−1 non-seizure records. The detector is then tasked with processing the N^{th} non-seizure record; the record that was withheld from the training set. This process was repeated N times so that each of the N non-seizure records is tested once. As a result of these two tests, the unary patient-specific detector was tested on the N+M records of a patient. The number of seizures missed, the average delay in declaring the electrical onset of detected seizures and the number of false detections was reported.

### Results

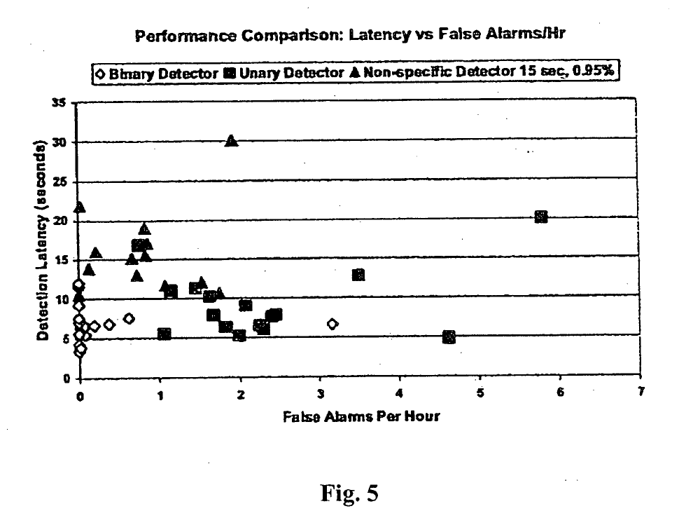

FIG. 5 illustrates how the three seizure detection methods perform in terms of seizure detection delay and false alarms per hour. Each data point on the graph represents a test subject. The optimal point on the performance plane is the origin {0 false alarms per hour, 0 second detection delay}. FIG. 5 shows that the binary patient-specific detector had the best mean performance coordinate {0.2+/−0.7 false alarm per hour, 6.8+/−2.4 seconds}.

However, if a low detection latency is valued over a low false detection rate, as may be the case in an application of seizure onset detection for the purpose of vagus nerve stimulation, then the unary patient-specific detector {2.3+/−1.3, 9.2+/−4.2} is favored over the patient non-specific detector {2.0+/−5.3, 17.8+/−10.0}. Three subjects on whom the non-specific detector performed particularly poorly are not shown in FIG. 5: subject 15 {0.19, 49.3}, subject 3 {0.15, missed all seizures}, subject 9 {22.0, 12.8).

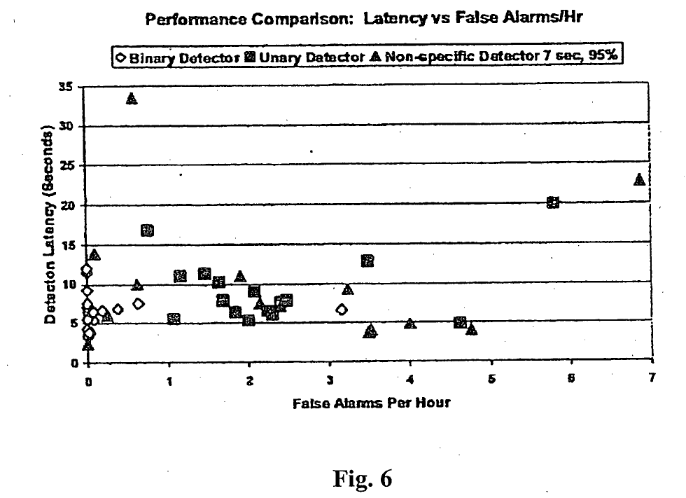

If the non-specific detector is biased towards detecting seizures earlier by choosing a configuration that uses a 95% confidence threshold and seven second segments, then detection latencies decrease and false-detection rates increase, as shown in FIG. 6. Two subjects on whom the non-specific detector performed particularly poorly are not shown: subject 3 {0.63, missed all seizures}, subject 9 {53.2, 4.6}.

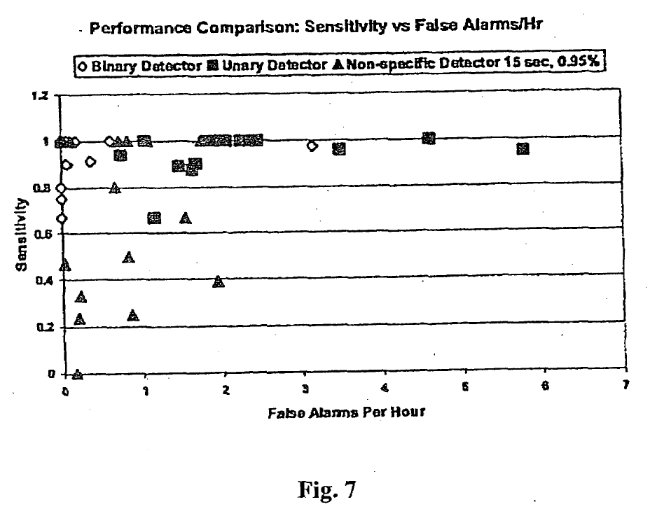

FIG. 7 illustrates how the three methods perform in terms of sensitivity (fraction of an individual's seizures that are detected) as well as false alarms per hour. The optimal point on the performance plane is the point {0 false alarms per hour, sensitivity of 1}. The binary patient-specific detector has the best mean performance coordinate {0.2+/−0.7 false alarms per hour, 0.93 sensitivity}. Again, depending on how one trades-off sensitivity for specificity, the unary patient-specific detector {2.3+/−1.3, 0.94} or the patient non-specific detector {2.0+/−5.3, 0.66} will turn out to be the right choice. One subject on whom the non-specific detector performed particularly poorly is not shown FIG. 6: subject 9 {22.0, 0.55}.

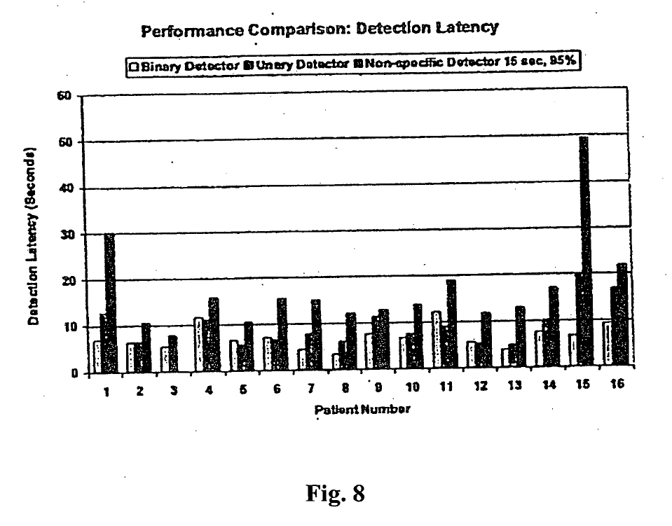

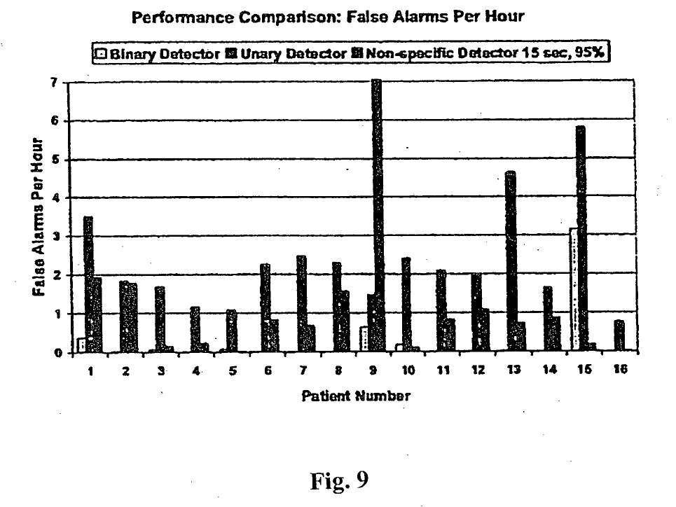

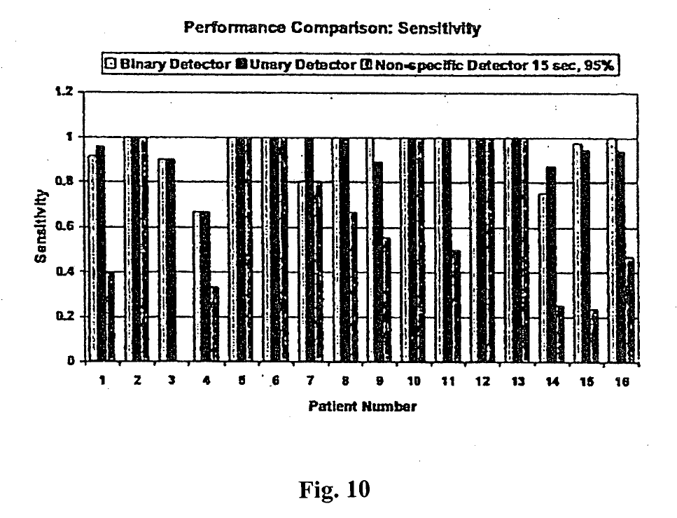

FIGS. 8-10 illustrate, for each patient, how well the seizure detection methods perform relative to each other. FIG. 8 depicts detector latencies; FIG. 9 depicts false detection rates; and FIG. 10 depicts detector sensitivities for each patient. The first column from the left is the binary detector, the second column represents the unary detector, and third column represents the non-specific detector.

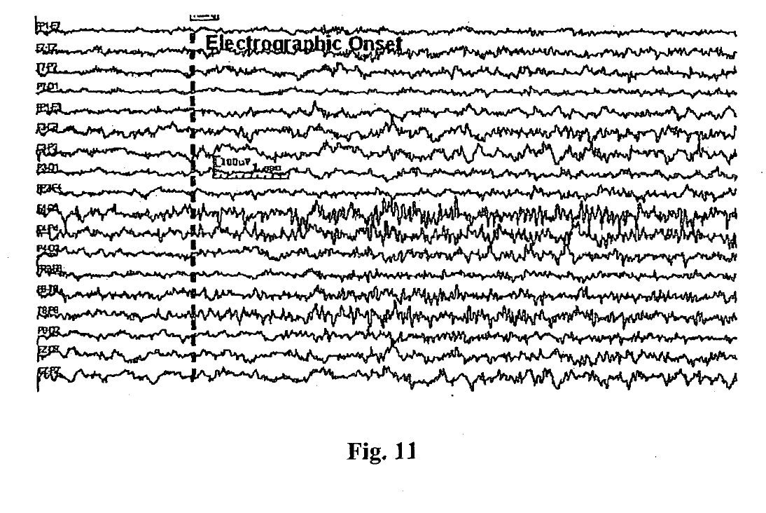

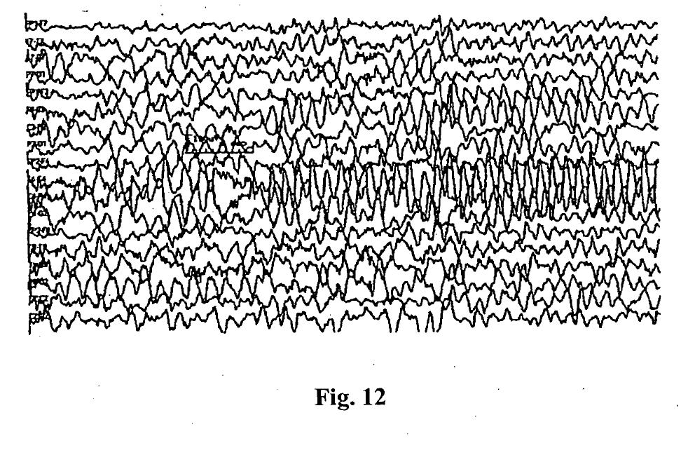

FIGS. 11-12 illustrate, on subject 1, the electrographic seizure state that is associated with the detection latency of each method. The focal electrographic onset of the subject's seizure is shown following the dotted line in FIG. 11. The binary detector declares that a seizure is ongoing during this focal phase, on average, 6.77+/−3.0 seconds after the electrographic onset. The unary detector also detects the focal phase, on average 12.8+/3.2 seconds after the electrographic onset. The patient non-specific detector declares that a seizure is ongoing during the generalized phase of the seizure, (illustrated in FIG. 12). The non-specific detector declares that a seizure is ongoing, on average, 30.1+/−15.4 seconds after the electrographic onset.

The finding that more patient-specific knowledge enhances the performance of a seizure detector stems from the fact that the detection problem becomes easier with more patient-specific information. To detect the electrical onset of a seizure, the binary patient-specific detector need only determine if features from an observed EEG waveform all fall within a small, specific region of the feature space referred to herein as the “seizure onset region.” This region's location is defined by an individual's seizure training data and its size defined by the individual's seizure training and non-seizure training data. Waveforms that look different from an individual's seizure onset (e.g. sleep, awake, interictal epileptiform bursts, artifact) are classified as non-seizure waveforms because they do not fall within the boundaries of the seizure onset region. This accounts for the high sensitivity and specificity demonstrated by the binary patient-specific detector.

The unary patient-specific detector faces a more difficult detection problem. The unary detector declares a seizure whenever features from an observed EEG waveform differ from features extracted from a non-seizure training EEG data set. As a consequence, any waveform that looks different from those in the training set triggers the detector; this includes the desired seizure waveforms as well as undesirable variants of awake, sleep, and artifact waveforms that may be underrepresented in the training set. This accounts for the unary patient-specific detector having a sensitivity that matches that of the binary but with worse specificity.

The patient non-specific detector faces the most difficult detection task. The non-specific detector declares a seizure whenever features from an observed EEG waveform resemble features extracted from archetypal seizures (i.e., non-patient specific). This approach works well for individuals whose seizure and non-seizure EEG conform to the archetypal patterns. On the other hand, this approach performs poorly on individuals whose seizures differ from archetypal seizures or whose non-seizure EEG demonstrates activity that resembles archetypal seizures. Without carefully examining the EEG of an individual and understanding how it relates to a set of archetypal seizures, few guarantees can be made about the performance of patient non-specific detector on a test individual. All this accounts for why the patient non-specific detector demonstrated lower performance relative to the binary patient-specific detector.

### Channel Reduction

According to one embodiment of the invention, the number of EEG channels necessary to detect a seizure may be reduced using a Recursive Feature Elimination (“RFE”) or other method to select the set of channels. [Reference: A patent is pending on RFE-SVM.] As explained in further detail below, the set of channels necessary to detect an epileptic seizure varies widely across patients. For some patients, embodiments having a one-channel detector may work as well as embodiments having a twenty-one-channel detector, and for others, embodiments having fifteen channels may be needed to attain performance comparable to that of a twenty-one-channel detector.

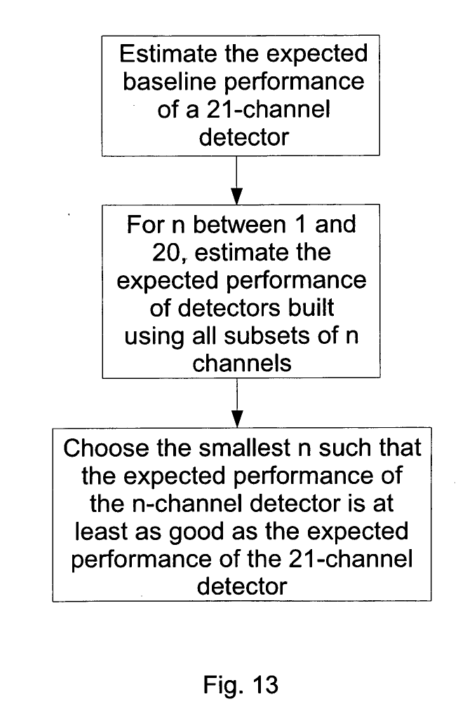

A brute force approach to determining the number of channels needed is outlined in FIG. 13. The underlying concept, according to one embodiment, estimates the expected performance of detectors using varying numbers of channels, and then chooses the smallest number of channels for which the expected performance is comparable to the expected performance of a twenty-one-channel detector. Unfortunately, this approach is computationally intractable since it involves training and testing on approximately 2²¹ different combinations of channels.

One embodiment of the invention to solve this problem includes a method that uses RFE to design SVM-based detectors that use small numbers of electrodes. The results presented below indicate that a surprisingly small number of electrodes (as few as two) often suffice to construct a detector that performs as well as detectors that use a full twenty-one channel montage.

According to one embodiment, an EEG-based, patient-specific seizure detector employs wavelet analysis to extract features from twenty-one channels of scalp EEG data and an SVM built using a radial basis function (RBF) kernel. Since the embodiment of the detector is patient specific, it is trained for a particular patient by training on data from that patient only.

According to one embodiment, step 2 of FIG. 1 is replaced by the following step:
2) For n between 1 and 20, use recursive feature elimination to choose the n best channels. Estimate the performance of the detectors built using those channels.
   The process of choosing n is described in more detail below.

The performance of a detector is evaluated in terms of its false positive (“FP”) rate, false negative (“FN”) rate, and latency. As discussed above false positive occurs when the detector declares a seizure outside of the window of time that the professional, who labeled the dataset, identified the seizure. A false negative occurs when the detector fails to declare a seizure at any time during the window of time that the professional identified as a seizure. The latency is the number of seconds between when the labeling professional marked a seizure onset and when the detector declared a seizure. In addition to FP, FN and latency, embodiments of the invention may estimate performance of a detector in terms of other criteria, such as energy consumption or any other metric.

Since there are 2²¹ different possible subsets that can be made from the original twenty-one channels, it is not practical to perform a brute-force, exhaustive search to find the subsets with which the detector obtains the best performance for a particular patient. Instead, RFE, a “greedy algorithm,” in one embodiment is used to choose a subset of each size that seems to provide the detector with sufficient information to perform well on future inputs. A version of RFE for non-linear SVM kernels is used since the RBF kernel is non-linear.

RFE uses, in one embodiment, the SVM machinery to rank the contributions of each channel in the set of channels being using for detection. Other ranking methods can be used within the RFE framework to rank the contribution of each channel. Once the RFE algorithm ranks the current set of n channels, the channel ranked as least important in the set is removed. This produces a set of n−1 channels. This rank-and-remove process is repeated on the set of n-channels which produces a set of n−2 channels. The process continues until one channel remains. When RFE is applied to a set of n channels, it produces a total of n−1 subsets. Though there is no guarantee that each subset found is indeed the best subset of that size, there are good reasons to believe that RFE finds one of the better subsets.

“Leave-one-out” cross validation is frequently used to estimate the generalization performance of classifiers built using machine learning. In one study, ten patients were analyzed resulting in a dataset that included, on average, 5.5 seizures per patient. According to those results, each seizure is embedded in a larger EEG stream that contains non-seizure EEG. For each patient, a “leave-one-seizure-file-out” cross validation methodology was used to evaluate the performance of the illustrative detectors built using various numbers of channels.

The leave-one-seizure-file-out process can be described as follows:

“Find average performance for full montage”
Init(aveAllPerf)
for s = each seizure in set of seizures S

C = all 21 channels
d = buildDetector (C, S − {s})
update(aveAllPerf, d, s)

end

The function update(avePer, d, s) calculates the performance of the detector d when used on the file s, and updates the measure of average performance avePer.

“Find min. number of channels with average performance at least as good
as aveAllPerf”
numNeeded = 21
for n = 20 to 1

init(aveSubsetPerf)
for s = each seizure in set of seizures S

S′ = S − {s}
C = RFE(n, S′) “Find n best channels”
d = build detector (C, S′)
update(aveSubsetPerf, d, s)

if aveSubsetPerf>= aveAllPerf

num needed = n

return numNeeded

The illustrative process listed above finds the smallest number of channels n, such that the average cross validation performance of detectors built using n channels is at least as good (with respect to each of the false negative rate, the false positive rate, and the latency) as the average cross validation for the twenty-one-channel detector. In other embodiments a less stringent selection criterion can be used. In the algorithm described above, the function buildDetector(C, S′) builds an SVM detector using the n channels in C while being trained on the files in S′. The function RFE(n, S′) finds the best n channels in the set of channels S′. S′ must contain at least n channels. The value aveAllPerf is the average performance of the detector when run using all channels on the set of seizures in S. aveSubsetPerf is computed using the average false positive rate, false negative rate, and latency for all of the detectors built using buildDetector for channel subsets of size n.

Importantly, the procedure finds the number of channels to be used, but does not directly compute which channels to use. The process does find a set of channels for each <size, cross validation set> pair, however RFE may find different channels for different cross validation sets in accordance with one embodiment.

Once the number of channels has been determined, RFE is run using all of the files in S to choose a set of channels. A detector is then trained on those channels and all of the files in S to create an ambulatory detector. The performance of the resulting detector is estimated by the average FP rate, FN rate and latency measured for all of the n-channel detectors built during leave-one-seizure-file-out cross-validation.

Because the data in a channel is the difference in scalp potential between two electrodes, the number of channels is not the same as the number of electrodes that would be necessary for the ambulatory detection system, since adjacent channels may share an electrode.

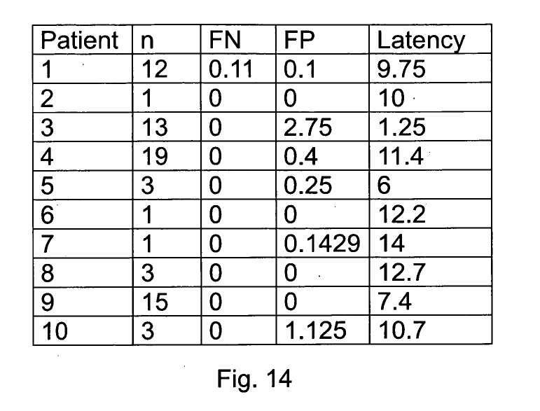

FIG. 14 presents a table showing, for each patient, the expected false negative rate, false positive rate, and latency derived for an embodiment of the n-channel detector. By construction, the n-channel detector performs at least as well as the twenty-one-channel detector. For some patients, the reduced channel detector performs slightly better in some respects than the twenty-one-channel detector, however the differences are not statistically significant.

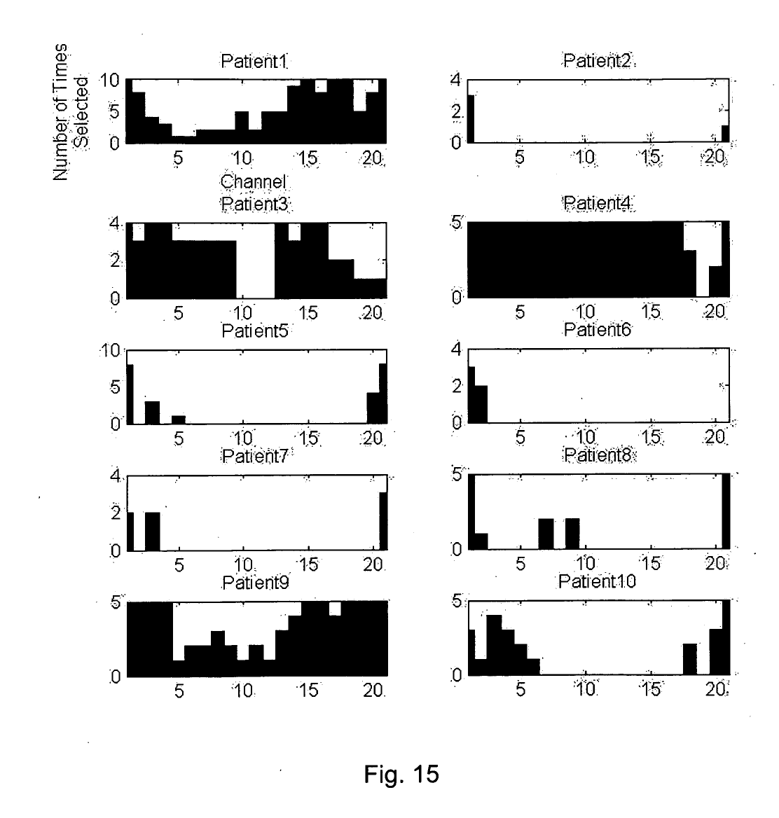

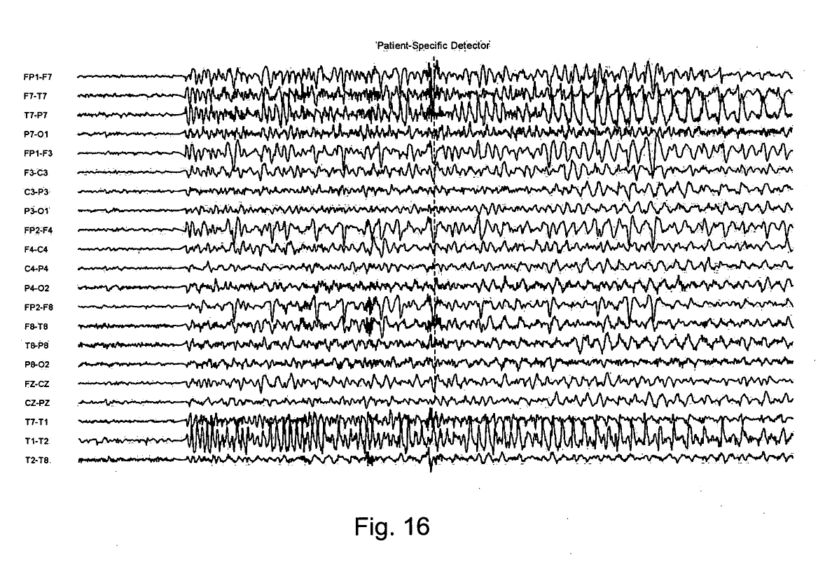

Different subsets of the data may lead to different choices of channels for the same patient. FIG. 15 shows how often each channel was chosen for each patient. For example, four seizure files were detected for patient number 2. For three of the leave-one-out tests RFE chose channel 1 (electrodes FP1 and F7), and for one of the tests it chose channel 21 (electrodes F7 and T7). FIG. 16 contains part of a seizure drawn from the EEG collected for patient number 2. The seizure has an abrupt and unmistakable onset during which channels 1 and 21 (the top two channels in FIG. 16) behave similarly. The illustrative process for building an n-channel detector for this patient chose a single channel, channel 1.

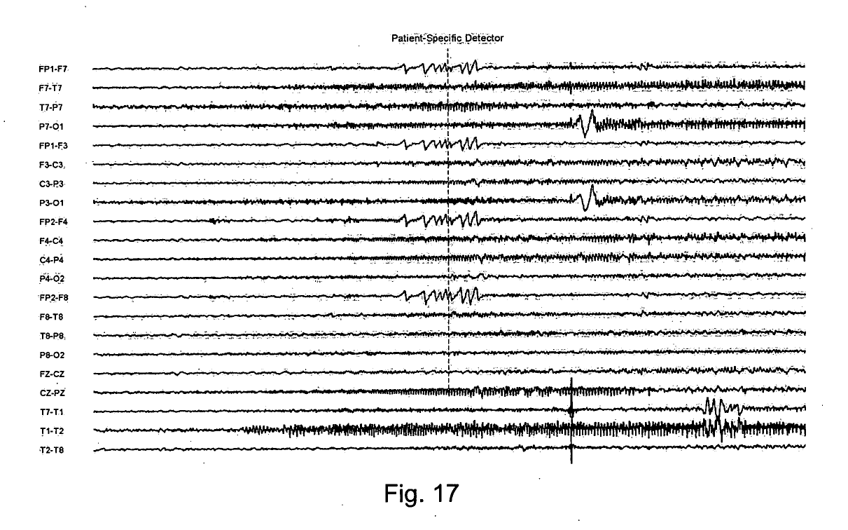

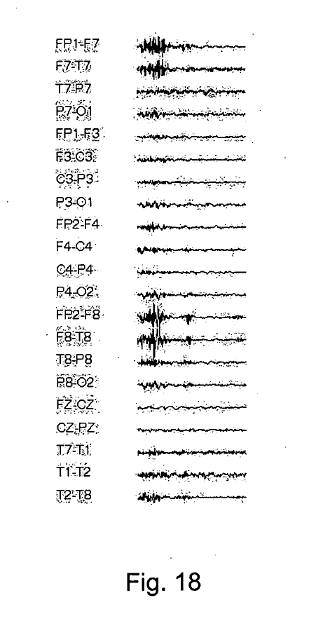

In general, for those patients requiring only a small number of channels, the channels cluster in the same region of the head. In contrast, for those patients for whom many channels are needed, e.g., patient number 9, the channels are typically widely dispersed. FIG. 17 contains part of a seizure drawn from the EEG collected for patient number 9. Even though fewer channels seem to be involved than for patient number 2, it requires more channels to reliably detect the seizure. This is because, at times, subsets of channels show seizure-like activity that does not evolve into a clinical seizure, as seen in FIG. 18. The illustrative method of building an n-channel detector for this patient chose fifteen channels involving eighteen of the twenty-one electrodes. The only channels not used were channels 5, 6, 7, 10, 11, and 12. This is consistent with what the histogram in FIG. 16 would lead one to expect.

While caution is taken in forming definitive conclusions from a study of a small amount of EEG data for ten patients, the data in FIG. 14 suggests that for some patients it is possible to perform epileptic seizure onset detection with a very small number of channels. According to one embodiment of the invention, for six out of the ten patients, as few as three channels are sufficient for detecting seizures of the types observed during testing.

The number of channels needed for a patient depends, not surprisingly, on characteristics of the patient's seizures. Some patients' seizures are focal in origin and consistently originate in a single small region of the brain. For those patients a small number of electrodes placed over the focus may be sufficient. For generalized seizures, in which seizure activity is present on most if not all electrodes, any electrode may be as good as any other, and again a small number of electrodes may be sufficient.

Some patients have different kinds of seizures with different origins. These patients will require more electrodes. Additionally, some channels may be naturally noisier than others or may produce confounding data (e.g., inter-ictal bursts that do not lead to clinical seizures). In such cases, more channels may be necessary to promptly discriminate seizures from other activities.

### Channel Addition

In another embodiment of the invention an algorithm uses machine learning to select a set of EEG channels to build a screening detector. In this embodiment and in contrast to the methods described above using recursive feature elimination, this embodiment of the invention utilizes a “recursive feature addition” method in which selected channels are added to a subset of channels incrementally based on the most useful channels.

A subset of channels is chosen based on how well a learning algorithm using various subsets performs on unseen data. An illustrative algorithm uses the original detector, D_{orig}, and a set of data as input. If F is the set of channels, the channel selection process can be described abstractly as:

| | |
|---|---|
| 1. | Create a set S of pairs where each pair consists of training data and |

test data. The training data and test data are subsets of the original data.

| 2. | For each subset f of channel set F, |
|---|---|
| | For each pair s ∈ S, |

a) Build detector D′ using training data of s and channels in f.
b) Get performance of D′ on test data of s.

| | |
|---|---|
| 3. | Select the best channel subset f′ using performance data. |
| 4. | Train the final detector using all data and channels f′. |

In Step 1, the training and test subsets are formed from the available data. One common way to evaluate a learning algorithm on available data is to remove one sample from the data set and train on the rest of the samples. The algorithm's performance can then be tested on the removed sample. This leave-one-out approach can be used to generate elements of the set S.

In Step 2, a detector is constructed using machine learning. In Step 2a, the training data is labeled using D_{orig}. The features extracted from the channels in subset f are used to form a new detector. The performance of this detector is estimated. Performance can be estimated in many ways. In this embodiment the screening detector is combined with D_{orig} to form a new detector D′ is created. The procedure is repeated for all elements of set S.

Using the performance data acquired in Step 2, the best subset can be chosen using appropriate criteria. Once the subset f′ is chosen, a detector that uses the channel subset is trained using all the available training data.

The approach described above is an exhaustive, brute-force approach. Given m channels, there are 2^{m} possible channels and thus this approach is impractical. Instead of examining all subsets, a greedy approach may be used which still uses machine learning to build the detectors, but the decision of which subsets to evaluate is performed incrementally. In a forward selection process, the best single channel subset is first chosen. Essentially, all possible single channels are tried and the performance of each single channel detector is estimated using unseen data. The best single channel subset is selected from all possible single channel subsets based on a selection criteria. Next, one of the remaining m−1 channels is added to the single channel subset and the best two channel subset is found. This process is repeated until there is a set of m−1 channel subsets. From these channel subsets, subset f′ is selected from which to build the final detector using performance metrics and a selection function. Alternatively, the process can be repeated until a set of stopping criteria is met. Criteria that may be used to determine performance of the detector may include, without limitation, FP, FN, latency, energy consumption, sensitivity, specificity or other measurements.

According to one trial, data from thirteen patients with seizures was collected. For each patient, data obtained from a previously trained patient-specific detection algorithm that operates as described above (the original detector) and a collection of files containing EEG data collected in a previous study were used. The length of the files varied from 2 to 30 minutes and each file contained one seizure.

In this embodiment, the selection function relates to the specificity, sensitivity, and energy consumption of the detector. The selection function is chosen in order to reduce the energy consumption of a multi-channel detector. The detector, denoted herein as “the screening detector,” is constructed using the channel subset and combined with the original detector to reduce energy consumption. A forward selection approach was used to perform channel selection for the screening detector. Thus, channel subsets are built as the algorithm is run. For each patient, training and test data pairs were generated using the leave-one-out approach described above. Since the data was already divided into files, each file was treated as a unit. Thus, for a patient with F files, F different training-test file pairs were present. For each screening detector built, a combined detector was formed and then executed on the appropriate test file to obtain false negative rates and cost information for each channel subset. Labeling performance was determined by comparing the labels output by an original detector, D_{orig}, on the test file to the labels output by the combined detector. The cost of the combined detector can be described as:

C=NₛCₛ+NₒCₒ+N_{b}C_{b}

where Cₛ, Cₒ and C_{b} are the costs of the screener, the original detector, and both detectors respectively. Nₛ, Nₒ, and N_{b} represent how many times the screener, the original detector, and both detectors are called. A separate term was introduced for both detectors because, in general, when idle time is accounted for, Cₒ+Cₛ≠C_{b}.

According to one embodiment, the best channel subset was selected by finding the subset that allows the training of a screening detector that when combined with the original detector has the lowest average cost. Moreover, none of the individual combined detectors should have a false negative rate greater than 0.25. This value based on the following analysis: Assume a seizure of length N. To detect a seizure, 3 consecutive positive windows must be found. Therefore the probability a seizure is missed is:

Pr_{[miss]}=α^{N-3+1}

where α is the false negative rate or the probability of mislabeling a single window. If N is chosen to be the minimum length of a seizure from the data set (i.e., 9) and choose 0.001 as the acceptable probability of missing a seizure, α=0.32.

While α=0.32 is an acceptable value for the false negative rate, a smaller value was chosen, in one embodiment, to increase the chance of detecting a seizure. Moreover, since the expected detection latency is

$$\frac{1}{1 - α} - 1$$

windows, a smaller a will lower the latency as well.

While the invention has been described with reference to illustrative embodiments, it will be understood by those skilled in the art that various other changes, omissions and/or additions may be made and substantial equivalents may be substituted for elements thereof without departing from the spirit and scope of the invention. In addition, many modifications may be made to adapt a particular situation or material to the teachings of the invention without departing from the scope thereof. Therefore, it is intended that the invention not be limited to the particular embodiment disclosed for carrying out this invention, but that the invention will include all embodiments falling within the scope of the appended claims. Moreover, unless specifically stated any use of the terms first, second, etc. do not denote any order or importance, but rather the terms first, second, etc. are used to distinguish one element from another.

1. A method for selecting a patient-specific subset of electrodes from a plurality of m EEG channels needed to detect an epileptic seizure in the patient, the method comprising the steps of:
collecting seizure EEG data from the plurality of m EEG channels;
selecting an effective subset n of the channels of the plurality of m EEG channels;
constructing a detector in response to the subset n of channels; and
estimating the performance of the detector in detecting seizures.

2. The method of claim 1 wherein the step of selecting the effective subset n of the channels of the plurality of m EEG channels comprises recursive feature elimination.

3. The method of claim 2 wherein recursive feature elimination comprises the steps of:
a. constructing a detector using the plurality of m channels;
b. estimating the performance of the detector;
c. removing a least useful channel from the plurality of m channels;
d. estimating the performance of the remaining plurality of channels;
e. repeating steps c and d until the performance of the remaining plurality of channels satisfies a criterion; and
f. setting n equal to the number of channels in the plurality of channels equal to one more than the number of channels in the plurality of channels that satisfied the criterion.

4. The method of claim 3 wherein the criterion comprises the performance of the remaining plurality of channels being worse than the performance of the plurality of m channels.

5. The method of claim 1 wherein the step of selecting the effective subset n of the channels of the plurality of m EEG channels comprises recursive feature addition.

6. The method of claim 5 wherein recursive feature addition comprises the steps of:
a. constructing a detector using one of the plurality of m channels;
b. estimating the performance of the detector;
c. adding a most useful channel from the plurality of m channels to the subset n;
d. estimating the performance of the plurality of channels in subset n;
e. repeating steps c and d until the performance of the plurality of channels in subset n satisfies a criterion; and
f. setting n equal to the number of channels in the plurality of channels that satisfied the criterion.

7. The method of claim 6 wherein the criterion comprises the performance of the remaining plurality of channels being no worse than the performance of the plurality of m channels.

8. The method of claim 1 wherein estimating the performance of the detector comprises evaluating at least one of the group consisting of false positive rate, false negative rate and latency.

9. The method of claim 1 wherein the step of estimating the performance of the detector is done with a cross-validation methodology.

10. The method of claim 1 wherein the detector is a support vector machine based detector.

11. The method of claim 10 wherein the support vector machine based detector comprises a radial basis kernel.

12. The method of claim 11 wherein the radial basis kernel is non-linear.

13. A patient-specific epileptic seizure detector comprising:
a plurality of electrodes corresponding to a plurality of m EEG channels;
a processor configured to select a subset n of the channels of the plurality of m EEG channels using recursive feature elimination, the detector constructed in response to the subset n of channels; and
an estimator configured to estimate the performance of the detector in detecting seizures.

14. The detector of claim 13 wherein the subset n comprises the plurality of m channels minus a plurality of least useful channels, whereby the least useful channels are determined by recursively removing the least useful channel from the plurality of m channels and estimating the performance of the remaining plurality of channels until the performance of the remaining plurality of channels satisfies a criterion, the subset n equal to the number of channels in the plurality of channels equal to one more than the number of channels in the plurality of channels that satisfied the criterion.

15. The detector of claim 13 wherein the estimator estimates the performance of the detector from at least one of the group consisting of a false positive rate, a false negative rate and latency.

16. The detector of claim 13 wherein the detector is a support vector machine based detector.

17. The detector of claim 16 wherein the support vector machine based detector comprises a radial basis kernel.

18. The detector of claim 17 wherein the radial basis kernel is non-linear.

19. A patient-specific epileptic seizure detector comprising:
a plurality of electrodes corresponding to a plurality of m EEG channels;
a processor configured to select a subset n of the channels of the plurality of m EEG channels using recursive feature addition, the detector constructed in response to the subset n of channels; and
an estimator configured to estimate the performance of the detector in detecting seizures.

20. The detector of claim 19 wherein the subset n comprises the plurality of m channels minus a plurality of least useful channels, whereby the subset n is determined incrementally by adding a most useful channel from the plurality of m channels and estimating the performance of the channels in the subset n until the performance of the channels in the subset n satisfies a criterion, the subset n to the number of channels in the plurality of channels that satisfied the criterion.
